(19) United States
(12) Patent Application Publication
(10) Pub. No.: US 2022/0061677 A1
(43) Pub. Date: Mar. 3, 2022

(54) PHYSIOLOGICAL MEASUREMENTS USING PHONE SCREEN

(71) Applicant: X Development LLC, Mountain View, CA (US)

(72) Inventors: Matthew Bennice, San Jose, CA (US); Anupama Thubagere Jagadeesh, Palo Alto, CA (US); David Andre, San Francisco, CA (US)

(21) Appl. No.: 17/382,055

(22) Filed: Jul. 21, 2021

Related U.S. Application Data
(60) Provisional application No. 63/070,684, filed on Aug. 26, 2020.

(51) Int. Cl. A61B 5/0205; A61B 5/00

(52) U.S. Cl. CPC A61B 5/0205; A61B 5/742; A61B 5/0077; A61B 5/6898

(57) ABSTRACT

A phone may be used to conduct physiological measurements such as heart rate, respiration rate, and arterial oxygen saturation level measurements. A mobile app may be installed on a user's portable electronic device, and may direct the user to place a part of the user's body onto a user-facing optical detector such as a camera. The portable electronic device may transmit at least two light signals to the body part using the portable electronic device's screen as an emission source. Reflections of the light signals are recorded by the optical detector. Based on the reflected light signal, the portable electronic device may determine the absorption of different light frequencies and the physiological parameter values.

### CROSS-REFERENCE TO RELATED APPLICATION

This application claims priority under 35 U.S.C. 119 to Provisional Application No. 63/070,684, filed Aug. 26, 2020, which is incorporated herein by reference.

### FIELD

This disclosure generally relates to electronic devices for conducting physiological measurements.

### BACKGROUND

People are often unable to perform timely diagnostic tests or monitor their health, e.g., physiological metrics such as oxygen saturation levels, to determine whether they have a serious medical condition or require hospitalization. The inability to monitor their health or perform tests may be due to sold out medical supplies or the lack of a simple, effective, and reliable method for monitoring their health while staying at home. In some cases, a lack of access to a doctor may prevent a sick person from being able to perform diagnostic or monitoring tests in a timely manner.

### SUMMARY

This specification describes systems, methods, devices, and other implementations for monitoring user physiological parameter values using a portable electronic device such as the user's phone.

In particular, a mobile app may be installed on a user's portable electronic device to conduct physiological measurements such as heart rate, respiration rate, and arterial oxygen saturation level measurements. The mobile app may direct the user to place a part of the user's body, e.g., the finger, onto a user-facing optical detector such as a camera. After the user's body part is placed on the optical detector, the portable electronic device may transmit at least two light signals to the body part using the portable electronic device's screen as an emission source. Reflections of the light signals are recorded by the optical detector. Based on the reflected light signal, the portable electronic device may determine the absorption of different light frequencies and the physiological parameter values. Other sensors on the portable electronic device may be used to continue ambient detection, e.g., to listen for coughs.

In general, innovative aspects of the subject matter described in this specification can be embodied in a portable electronic device that includes a display, a camera, and a processor. The display includes one or more emitters that are configured to transmit a first transmission signal at a first wavelength onto a body part of the user and a second transmission signal at a second wavelength onto the body part. The camera is configured to obtain one or more images comprising a first reflection signal in response to the first signal being transmitted onto the body part and a second reflection signal in response to the second signal being transmitted onto the body part. The processor is configured to determine one or more physiological parameter values of the user using the one or more images obtained by the camera, and the body part is a finger of the user.

In some implementations, the first wavelength corresponds to a red light and the second wavelength corresponds to a green light or a blue light.

In some implementations, the one or more emitters include a first emitter, a second emitter, and a third emitter. The first emitter is configured to emit the first transmission signal at the first wavelength, which corresponds to red light. The second emitter is configured to emit the second transmission signal at the second wavelength, which corresponds to green light. The third emitter is configured to emit the third transmission signal at the third wavelength, which corresponds to blue light.

In some implementations, the one or more emitters comprise a first emitter configured to emit the first transmission signal, the second transmission signal, and a third transmission signal. The first transmission signal corresponds to a red light at a first time. The second transmission signal corresponds to a green light at a second time different from the first time. The third transmission signal corresponds to a blue light at a third time that is different from the first time or the second time.

In some implementations, the one or more emitters include one or more light emitting diodes. The display is configured to display the one or more physiological parameter values. The one or more physiological parameter values comprise one or more values for a heart rate, a respiration rate, an arterial oxygen saturation level, an iron level, a glucose level, a ketone level, a urea level, and a creatine kinase level.

In some implementations, the portable electronic device also includes a detector configured to detect a presence of a body part of the user within a threshold distance of the display.

The one or more emitters are configured to emit the first transmission signal and the second transmission signal in response to detecting the presence of the body part of the user within the threshold distance of the display.

In some implementations, the processor is configured to determine whether each of a signal to noise ratio of the first reflection signal and a signal to noise ratio of the second reflection signal satisfy a threshold. In response to determining that the signal to noise ratio of the first reflection signal does not satisfy the threshold, the one or more emitters are configured to retransmit the first transmission signal at a greater light emission level than an emission level of the prior transmission of the first transmission signal.

In some implementations, the processor is configured to determine whether each of a signal to noise ratio of the first reflection signal and a signal to noise ratio of the second reflection signal satisfy a threshold. In response to determining that the signal to noise ratio of the second reflection signal does not satisfy the threshold, the one or more emitters are configured to retransmit the second transmission signal at a greater light emission level than an emission level of the prior transmission of the second transmission signal.

According to some implementations, innovative aspects of the subject matter described in this specification can be embodied in a method of obtaining a physiological parameter value of a user using a portable electronic device. The operations include receiving, by a processor in the portable electronic device, an initiation signal to initiate measurement of the physiological parameter value of the user. In response to receiving the initiation signal to initiate the measurement of the physiological parameter value of the user, one or more emitters in a display of the portable electronic device are controlled to transmit a first transmission signal at a first wavelength and a second transmission signal at a second wavelength onto a body part of the user. One or more images including a first reflection signal is obtained in response to the first signal being transmitted onto the body part and a second reflection signal in response to the second signal being transmitted onto the body part. The processor in the portable electronic device determines the physiological parameter value using the one or more images. The display of the portable electronic device displays data indicative of the physiological parameter value.

In some implementations, in response to receiving the initiation signal to initiate the measurement of the physiological parameter value of the user, the processor determines whether the body part of the user is within a threshold distance of a camera of the portable electronic device. The one or more emitters in the display of the portable electronic device are controlled to transmit the first transmission signal and the second transmission signal in response to determining that the body part of the user is within the threshold distance of the camera of the portable electronic device.

In some implementations, when multiple emitters are configured to emit light signals, obtaining the one or more images including the first reflection signal in response to the first signal being transmitted onto the body part and the second reflection signal in response to the second signal being transmitted onto the body part comprises obtaining a still image or a video that captures the first reflection signal and the second reflection signal.

In some implementations, when a single emitter is configured to emit light signals, obtaining the one or more images including the first reflection signal in response to the first signal being transmitted onto the body part and the second reflection signal in response to the second signal being transmitted onto the body part comprises obtaining multiple sequential images or a video that capture the first reflection signal and the second reflection signal.

In some implementations, displaying the data indicative of the physiological parameter value includes displaying data indicative of at least one of a heart rate, a respiration rate, and optically sensitive molecular signals within the blood that are optically sensitive such as an arterial oxygen saturation level, an iron level, a glucose level, a ketone level, a urea level, and a creatine kinase level.

In some implementations, the method also includes determining whether each of a signal to noise ratio of the first reflection signal and a signal to noise ratio of the second reflection signal satisfies a threshold. In response to determining that the signal to noise ratio of the first reflection signal does not satisfy the threshold, the first transmission signal is retransmitted at a greater light emission level than an emission level of the prior transmission of the first transmission signal. In response to determining that the signal to noise ratio of the second reflection signal does not satisfy the threshold, the second transmission signal is retransmitted at a greater light emission level than an emission level of the prior transmission of the second transmission signal.

According to some implementations, a non-transitory computer-readable storage medium includes instructions, which when executed by one or more processors in a portable electronic device, cause the one or more processors to execute operations. The operations include receiving an initiation signal to initiate measurement of the physiological parameter value of the user. In response to receiving the initiation signal to initiate the measurement of the physiological parameter value of the user, one or more emitters in a display of the portable electronic device are controlled to transmit a first transmission signal at a first wavelength and a second transmission signal at a second wavelength onto a body part of the user. An image including a first reflection signal is obtained in response to the first signal being transmitted onto the body part and a second reflection signal in response to the second signal being transmitted onto the body part. A physiological parameter value of the user is determined using the image. The display of the portable electronic device is controlled to display data indicative of the physiological parameter value.

Other aspects include corresponding methods, systems, apparatus, computer-readable storage media, and computer programs configured to implement the operations of the above-noted methods.

The above-noted implementations and description below provide several advantages and conveniences for users. For example, users may monitor their health at home without having to purchase additional medical devices and without having to visit a doctor's office. A doctor can ask a patient or the patient's family member to obtain physiological measurements and track the patient's health over a period of time. Obtained data may be shared with the doctor if the patient consents to sharing this data. By eliminating the need for a person infected or potentially infected by a virus to visit the doctor's office, potential viral transmission to members of the public and staff at the doctor's office can be prevented.

A significant benefit of the systems, devices, and methods for obtaining physiological parameter values described in this disclosure is that the user does not have to incur the cost and inconvenience of having to buy additional devices or traveling to a pharmacy to purchase equipment to perform tests. Instead, the user can use their phone and, through the convenience of a mobile application installed on the phone, can obtain physiological parameter values simply by placing a part of the user's body, such as the finger, on the phone.

With increased and more convenient monitoring and obtaining of the patient's oxygen saturation levels, informed and timely decisions can be made by the patient, the patient's family, or the patient's doctor as to whether the patient needs advanced care such as hospitalization or to be connected to a ventilator. This remote patient health monitoring method can be particularly advantageous in countries or remote areas where health care is not as readily accessible.

Additionally, data obtained through the mobile application can be anonymized and aggregated to generate additional information on viruses. For example, the anonym ized and aggregated data can be used to generate heat maps or provide real time indications of virus flare-ups and/or regions with a high density of infected population.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts an example portable electronic device for measuring physiological parameter values of a user in accordance with implementations of the present disclosure.

FIG. 2 depicts an example block diagram of a portable electronic device for obtaining physiological parameter values of a user according to implementations of the present disclosure.

FIG. 3 depicts an example setup of a reflection pulse oximetry measurement according to implementations of the present disclosure.

FIGS. 4A-4B depict example measurements of red and green detected light according to implementations of the present disclosure.

FIG. 5 depicts a flowchart of an example process to obtain physiological parameter values of a user using a portable electronic device according to implementations of the present disclosure.

FIGS. 6A-6G depict example screen shots of a mobile application when executing a method to obtain physiological parameter values according to implementations of the present disclosure.

Like reference, numbers, and designations in the various drawings indicate like elements.

### DETAILED DESCRIPTION

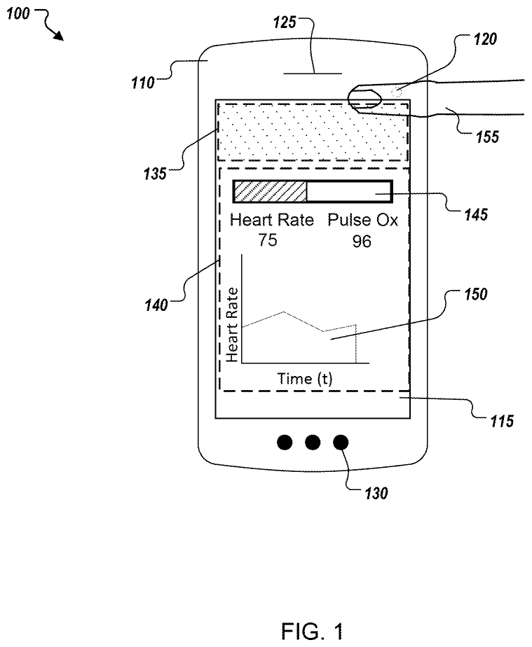

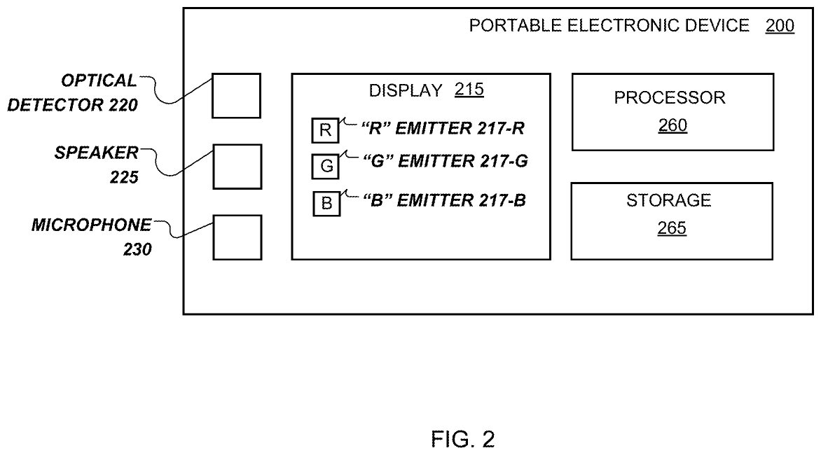

FIG. 1 depicts an example portable electronic device 100 for measuring physiological parameter values of a user. The portable electronic device 100 may include a housing 110 and a display 115. The housing 110 is a shell that protects various components of the portable electronic device 100 such as processors, memory, optical detector 120, speaker 125, microphone 130, and various other electronic circuitry. Speaker 125 and microphone 130 are described in more detail below with respect to FIG. 2.

The optical detector 120 is configured to detect one or more light signals from the environment that the portable electronic device 100 is located in. In some implementations, the optical detector 120 may be a camera. The camera may be configured to obtain still or moving images in response to light emitted from the display 115 as described in more detail below. In some implementations, the optical detector 120 may be activated in response to a finger detector in the portable electronic device 100 detecting a presence of a finger 155 of the user. In some implementations, the optical detector 120 may be activated in response to a touch sensor configured to detect a touch corresponding to an input of the finger 155.

The portable electronic device 100 may execute an application for monitoring physiological measurements. The user may download the application through various methods, e.g., from an application store, the Internet, a disk drive, or a server. After installing the application, the user may open the application in the portable electronic device 100 whenever the user wants to obtain physiological parameter values.

The portable electronic device 100 may open the application when one or more buttons on the portable electronic device 100 are selected by the user or when the portable electronic device 100 receives an audio signal indicating that the user would like to open the application.

When the application is open, the display 115 may display a user interface on the display screen to facilitate user interaction with the application. The display 115 may include one or more emitters to emit light at different wavelengths. Various suitable types of emitters may be used, including, but not limited to, light emitting diodes (LEDs).

A screen of the display 115 may be divided into multiple sections. For example, in FIG. 1, the screen is divided into an emission portion 135 and a graphical user interface (GUI) portion 140. The emission portion 135 is configured to emit light at one or more wavelengths, such as a red light and/or a green light. The emission portion 135 may be a portion of the screen that is located closest to the optical detector 120.

The display 115 may be configured to activate the emission portion 135 when a measurement is to be obtained. When activated, the emission portion 135 may emit one or more light signals using the emitters in display 115. When a measurement is not to be obtained or after a measurement has been completed, the emission portion 135 may be deactivated, and the GUI portion 140 may encompass the entire screen or both portions 135 and 140.

The GUI portion 140 may output various types of content. For instance, in some cases, the GUI portion 140 may display user instructions to initiate or complete a measurement. In some cases, the GUI portion 140 may display a status bar 145 indicating the status of a measurement being taken or task being performed. For instance, in FIG. 1, a status bar 145 shows a measurement progress of just under 50% to obtain measurements for a particular physiological parameter.

In some cases, the GUI portion 140 may display measured data such as a measured heart rate and/or arterial oxygen saturation level of the user. In some cases, the GUI portion may display aggregated data 150 that may include data measured over an extended period of time. The aggregated data 150 may be displayed in various suitable ways. In FIG. 1, a graph is used to depict the heart rate of the user over a period of time.

The GUI portion 140 may also be configured to receive input from the user. For example, the GUI portion 140 may include one or more sensors. e.g., capacitive touch sensors, that may detect a touch of the user when the display 140 includes a touch screen. The GUI portion 140 may display one or more fields, symbols, or icons, which may be manipulated or selected by a user when the user desires to provide an input.

FIG. 2 depicts an example block diagram of a portable electronic device 200. The portable electronic device 200 may correspond to the portable electronic device 100 shown in FIGS. 1, 3, and 6. In general, the portable electronic device 200 may correspond to any portable electronic device with a display screen, a camera, a processor, and memory. Examples of the portable electronic device 200 include, but are not limited to, a cellular phone, a mobile phone, a smart phone, a laptop, an electronic pad, or a personal digital assistant. The portable electronic device 200 may include a display 215, an optical detector 220, a speaker 225, a microphone 230, a processor 260, and storage 265.

The optical detector 220 corresponds to the optical detector 120 described above with respect to FIG. 1. The microphone 130 is configured to detect audio signals from the environment in which the portable electronic device 100 is located. For example, the microphone 130 may detect voice signals that include one or more instructions of a user. In some cases, the microphone 130 may be activated to listen to audio indicators of the user's health. For example, the microphone 130 may be activated to listen for user coughs and sneezes.

The speaker 125 may emit audio signals generated by the portable electronic device 100 or signals received from other devices to be communicated to the user. For example, the speaker 125 may output an audio signal that includes instructions for conducting one or more physiological measurement operations.

The display 215 may include one or more emitters configured to emit light at different wavelengths. In some implementations, the emitters may include, but are not limited to a red light emitter 217-R, a green light emitter 217-G, and a blue light emitter 217-B. The red light emitter 217-R is configured to emit a light having a wavelength of approximately 635-700 nm, which corresponds to light having a red color. The green light emitter 217-G is configured to emit a light having a wavelength of approximately 520-560 nm, which corresponds to light having a green color. The blue light emitter 217-B is configured to emit a light having a wavelength of approximately 450-400 nm, which corresponds to light having a blue color.

In some implementations, the emitters 217-R, 217-G, 217-B may be controlled to output a particular RGB value for a pixel. In some implementations, the emitters 217-R, 217-G, 217-B may be configured to output a particular type of light. For example, the emitters 217-R, 217-G, 217-B may be configured to output only red light, only green light, only blue light, only red and green light, only red and blue light, or only green and blue light. In some implementations, only emitters 217-R, 217-G, 217-B located in an emission portion, e.g., emission portion 135, of display 215 may be activated to emit light when conducting a test to obtain a physiological measurement.

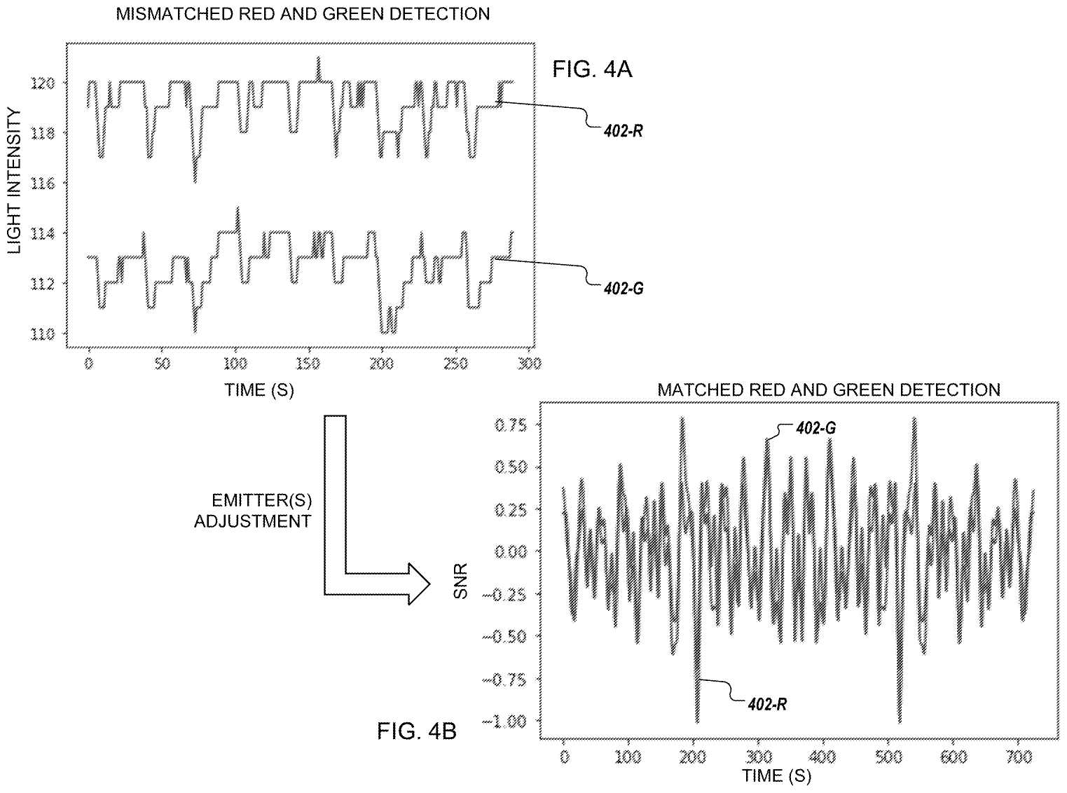

The emission and/or illumination levels of each of the emitters 217-R, 217-G, 217-B may be controlled if greater levels of one light compared to another light are desired. For example, as described in more detail below with respect to FIGS. 4A and 4B, because red, green, and blue light are absorbed differently by the human body, the emission levels of the emitters 217-R, 217-G, 217-B can be set differently to accommodate the absorption characteristics of the human body.

In some implementations, a single emitter may be used. The single emitter may be configured to emit light at different wavelengths, such as red light, green light, and blue light sequentially, i.e., at different sequential times.

The processor 260 may be coupled to all components of the portable electronic device 200, and may control the operations of the portable electronic device 200. The processor 260 may be a data processing apparatus that includes various logic circuitry and programs to execute the various implementations described herein. The processor 260 may execute one or more computer programs for executing the physiological measurement mobile application described in this disclosure.

The processor 260 may include, by way of example, both general and special purpose microprocessors. The processor 260 may receive instructions and data from an input/output port or device, read only memory, a random access memory, or a combination thereof. For instance, the processor 260 may be configured to execute instructions stored in a storage 265.

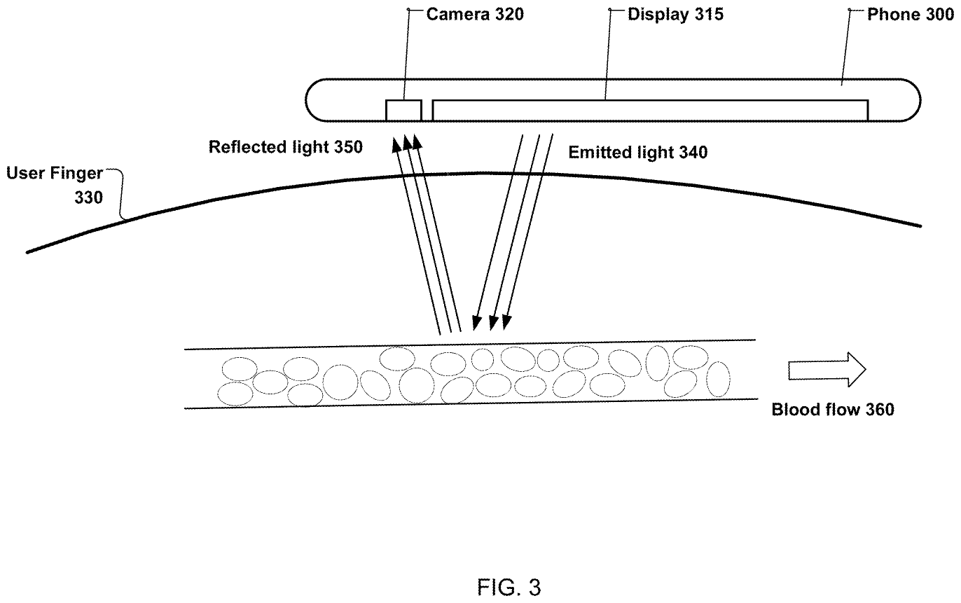

Storage 265 may include one or more mass storage devices, e.g., magnetic, magneto optical disks, optical disks, EPROM, EEPROM, flash memory devices, and may be implemented as internal hard disks, removable disks, magneto optical disks, CD ROM, or DVD-ROM disks for storing data. Storage 265 may include a read only memory or a random access memory or both. Storage 265 may include computer-readable media suitable for storing data and computer program instructions for executing operations of the portable electronic device 200. Data obtained by performing the physiological measurements described in this disclosure may be stored in storage 265. Details of the measurement process are provided below with respect to FIGS. 3-6.

FIG. 3 depicts an example setup of a reflection pulse oximetry measurement. In the example shown in FIG. 3, the portable electronic device 300 is a cellular or mobile phone 300. The phone 300 includes a display 315 and a camera 320, which correspond to the display 115, 215 and optical detector 120, 220 described above in FIGS. 1 and 2.

In FIG. 3, a side view is shown of user finger 330 next to a phone 300. When a physiological measurement is desired, the user may place the user's finger 330 in proximity of the camera 320. As an example, the user may place the portion of the user's finger 330 having the fingerprints proximity of the camera 320. In some cases, reflection pulse oximetry measurements from other parts of the body may be taken using the same technique. In such cases, the other body part is placed within proximity of the camera 320 instead of the user finger 330 shown in FIG. 3.

Various configurations for placing the finger 330 may be used as long as the finger 330 is located in an area where it can receive light emitted from the display 315 and light reflected from the finger 330 can reach the camera 320. For example, in some implementations, the finger 330 must be within a threshold distance and angle of the phone 300 to obtain physiological measurement data. The threshold distance may be variably set and may be zero or a distance set by the system designers. In some implementations, the finger 330 must be touching the phone 300 in a region above the camera 300. In some implementations, the finger 330 must be placed above a region between the display 315 and a camera 320 such that the finger 330 partially overlaps the display 315 and the camera 320.

When the finger 330 is in proximity of the camera 320, display 315 may emit two or more wavelengths of light 340 onto the user's finger 330. The emitted light 340 may scatter upon interacting with the finger 330. For instance, part of the emitted light 340 may reflect off the skin of the finger 330. Part of the emitted light 340 may traverse beyond the skin and may further scatter and interact with elements such as user tissue and blood particles within the user's finger 330.

A flow direction 360 of the blood particles is shown in FIG. 3. The blood particles may be of different shapes, sizes, and velocities. Pulse oximetry measurements may obtain a photoplethysmographic (PPG) signal from the reflected light 350 that is caused by variations in the quantity of arterial blood associated with periodic contraction and relaxation of a user's heart. The PPG signal magnitude may depend on the amount of blood ejected from the heart into the peripheral vascular bed with each systolic cycle, the amount of absorption by the blood, skin, and tissue, and the wavelengths of the emitted light.

The PPG signal is composed of a DC signal and an AC signal. The DC signal may reflect the light absorbed, scattered, and reflected by venous and capillary blood, and bloodless stationary tissues. The AC signal may reflect the light absorbed, scattered, and reflected by arterial blood. The heart rate and respiration data may be obtained by performing a frequency domain analysis on the AC signal. For instance, filtering and Fourier Transforms may be performed on one or more portions of the AC signal to remove noise and identify spectral peaks.

While several pulse oximeter systems rely on red and infrared (IR) light to determine the oxygen saturation levels in a user's blood, such measurements are problematic because most phone cameras are not sensitive to IR lights. In this disclosure, other wavelengths of light such as green or blue light are used instead of IR light. For example, a ratio of the detected light signal when two different types of light, e.g., red and green lights, are emitted may be used to calculate oxygen saturation levels in a user's blood. Implementations described below use the example of red and green emitted lights. However, other emitted lights, e.g., red and blue, or other colors, may also be used.

In more detail, the camera 320 may detect reflected light signals 350 from the user finger 330. The phone 300 may determine the AC and DC signals of the red PPG reflection signal that forms part of the light signals 350, and the AC and DC signals of the green PPG reflection signal that forms part of the light signals 350. The phone 300 may determine a ratio of the AC-to-DC signals for the red PPG reflection signal detected in response to the red light 340 being emitted. The phone 300 may also determine a ratio of the AC-to-DC signals for the green PPG reflection signal detected in response to the green light 340 being emitted. The phone 300 may then determine a ratio of the red AC-to-DC signal ratio to the green AC-to-DC signal ratio.

The determined ratio of AC to DC signals may reveal the oxygen saturation levels in the user's blood and is largely independent of the volume of arterial blood entering the tissue during systole, skin pigmentation, skin thickness and vascular structure. Oxygen saturation levels in a user's blood may indicate whether a user has respiratory illness.

For example, one of the symptoms experienced by a patient infected by respiratory illnesses is that the person's oxygen saturation levels drop due to the presence of fluid around pneumonia-infected lungs (pleural effusion). The reduction of oxygen levels in a person's body is also known as Hypoxemia. Patients without hypoxemia may have pulse oximeter readings ranging from 95 to 100 percent of the normal levels of oxygen in blood. Patients with hypoxemia may have pulse oximeter readings under 90 percent.

If a patient periodically or continuously monitors oxygen saturation levels using the pulse oximetry method described above, the patient may be able to determine when the patient's oxygen saturation levels have dropped substantially. The patient (or a doctor of the patient who has received the test results) may then determine that the patient should be hospitalized and can decide whether a ventilator should be provided to the patient. If appropriate, the patient or the patient's doctor may also take one or more actions such as calling for emergency ambulatory services to assist the patient.

In some implementations, calibration may be performed to improve the accuracy of the measurements. As explained above, red, green, and blue light are absorbed differently by the human body. For example, blue light is absorbed by the human body significantly more than red or green light. As a result, when blue light is emitted onto user tissue, the light signal that reflects back from the user's tissue may have a smaller magnitude compared to when red or green light is emitted onto the user tissue. The smaller magnitude is due to greater absorption by the user tissue. Another example is human skin, which absorbs red, green, and blue lights at different rates.

In general, human tissues or blood particles may have the same or different absorption rates for light emitted at particular wavelengths. For instance, oxygenated blood may absorb 12% as much red light as deoxygenated blood. However, oxygenated blood and deoxygenated blood have approximately the same absorption for green light. Due to the various measurement parameters that depend on tissue content and blood content that can vary from one person to another, a calibration technique may be applied to address these variations and improve the accuracy of the physiological measurements.

FIG. 4A depicts example raw signal outputs of the light intensities of a reflection signal 402-R detected in response to a red emitted light signal and a reflection signal 402-G detected in response to a green emitted light signal. The magnitude of reflection signal 402-R is substantially greater than the magnitude of reflection signal 402-G even though a higher emission level was used for the emitted green light. In this example, the signal-to-noise ratios (SNRs) of both reflection signals 402-R and 402-G are therefore substantively different, which may lead to measurement errors.

As part of the calibration method, the emission levels for the emitters in the display of a portable electronic device can be adjusted so that the obtained reflection signals 402-R and 402-G have similar magnitudes or SNRs. For instance, with respect to FIG. 4A, the emission level of the emitted green light may be increased as part of the calibration so that a satisfactory SNR can be obtained for the reflection signal 402-G. In some cases, the adjustment may involve decreasing the emission level of a particular emitted light. For instance, with respect to FIG. 4A, the emission level of the emitted red light may be decreased as part of the calibration so that the SNRs of both reflection signals 402-R and 402-G are almost the same.

The adjustment of light emission levels may be repeated multiple times as part of the calibration procedure until a satisfactory signal magnitude and SNR are obtained for a reflection signal. The light emissions levels may be controlled by adjusting the power of the emitters, e.g., emitters 217-R, 217-G, 217-B as shown in FIG. 2, in the display of the portable electronic device.

FIG. 4B depicts an example of the obtained reflection signals 402-R and 402-G after calibration has been performed. As can be seen in FIG. 4B, the SNRs of both reflection signals 402-R and 402-G is very similar. Such data can then be used to determine the physiological parameter values for the user.

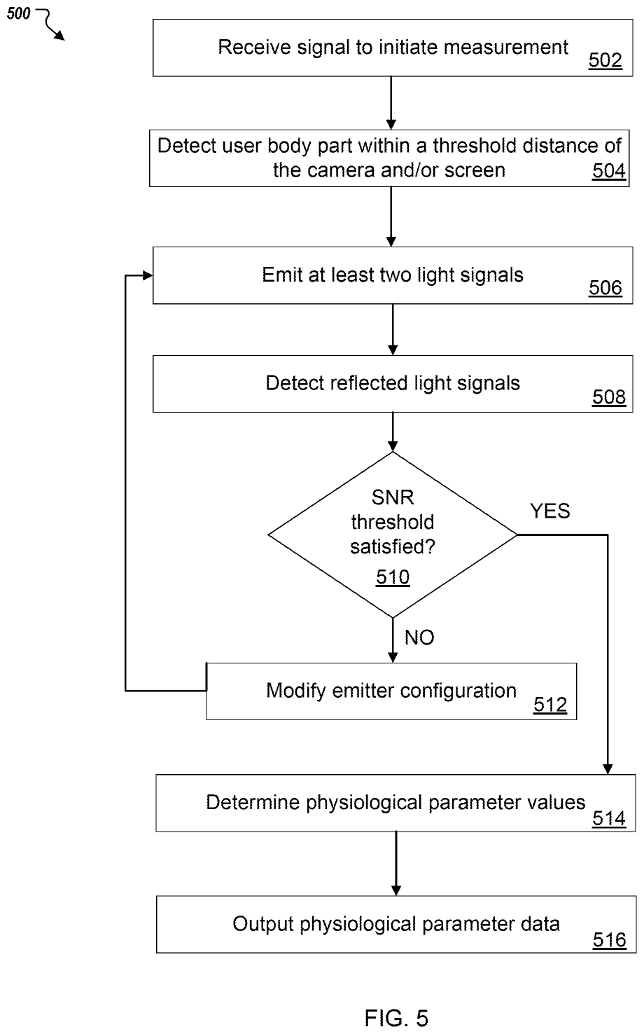

FIG. 5 depicts a flowchart of an example process 500 to obtain physiological parameter values of a user using a portable electronic device. The operations of FIG. 5 are described below as being performed by the portable electronic device. However, one, multiple, or all of the operations of process 500 may be implemented by or under control of one or more components of the portable electronic device such as a processor of the portable electronic device or under control of the processor.

The process may be initiated when the portable electronic device receives an initiation signal to initiate the measurements (502). One of several types of signals may serve as the initiation signal. For instance, in some cases, when the user selects a symbol or icon corresponding to the mobile application for obtaining physiological measurements on the display of the portable electronic device, the portable electronic device may detect the selection, e.g., through a sensor, interpret the selection or the signal generated by the sensor in response to the selection as a request to initiate the measurements. The portable electronic device may then open the mobile application and display a message asking the user to place a part of the user's body, e.g., finger, in proximity to the optical detector, e.g., camera, of the portable electronic device, as described above.

In some cases, when the mobile application is open, the user may instruct the portable electronic device to initiate a physiological measurement. The instructions may be provided through various suitable means such as a voice command of the user, a selection of a button or icon in the mobile application's user interface configured to initiate the physiological measurement, or a physical gesture of the user or movement of the portable electronic device. The portable electronic device may be preprogrammed to interpret particular gestures and device movement as a request to initiate a physiological measurement.

In some implementations, after receiving the signal to initiate measurements, the portable electronic device may display one or more messages and may request input from the user. For instance, in some cases, the portable electronic device may display instructions on how to perform the measurement as a guide to assist the user in conducting the measurement. The portable electronic device may display a message before or after any operation to indicate to the user what the next operation is or when an operation has been completed. As an example, the portable electronic device may display a message asking the user to place the user's finger in proximity to the optical detector. When the measurements are being conducted and light is being emitted, the portable electronic device may display a message or status bar indicating that the measurement is in progress and for the user to remain still, e.g., not move the user's finger, while the measurement is being conducted.

In some cases, the portable electronic device may display a message indicating that the user's personal information may be obtained as a result of performing the measurement. The portable electronic device may request authorization from the user to obtain and store the user's personal information including the measurement results. In some cases, the portable electronic device may provide options for the user to configure the collection of the user's personal information. For instance, the portable electronic device may query the user if the user approves anonym ization and/or aggregation of the user's measurement results. If the user approves anonym ization and/or aggregation of the user's measurement results, the portable electronic device may strip user data of personal information such as name, address, or other information that may identify the user. The stripped information may then be aggregated or shared with other approved entities.

In another example, the portable electronic device may query the user to determine if the user would like to share the user's measurement results with certain entities or people. For instance, the portable electronic device may ask the user whether the user would like to share the user's measurement results with other entities or people such as a doctor, friend, family member, insurance company, or primary care physician. If the user responds affirmatively, the portable electronic device may then display an interface through which the user can import or provide the contact information of the entities or people with whom the user has approved sharing the measurement results. The user may also indicate how often or when results should be shared with one or more of the entities.

In another example, the portable electronic device may query the user for additional information regarding the user's health. For example, the portable electronic device may ask the user to input the user's body temperature, blood pressure, cholesterol levels, or other biometric data. Such information may be helpful, e.g., to determine if a user is displaying multiple symptoms of a particular disease. As an example, high fever is a common symptom of several diseases, pandemics, and viral infections. By requesting temperature information and determining the oxygen saturation levels in the user's blood as described in this disclosure, the portable electronic device may be able to provide additional information to the user or the user's doctor regarding the symptoms experienced by the user.

In some implementations, the portable electronic device may activate one or more sensors and devices to obtain additional information on the user's health. For example, the portable electronic device may activate a microphone for a particular time period to detect how many times a person may be coughing during the particular time period.

In some implementations, after receiving the signal to initiate measurements (502), the portable electronic device may wait until a body part of the user is detected within a threshold distance of the optical detector and/or the screen of the portable electronic device. In general, various parts of the user's body may be tested to obtain physiological parameter values of the user. For the purposes of this example, a finger is used as the body part from which physiological measurements are obtained.

As explained above, the portable electronic device may provide audio and/or visual instructions to the user to place the user's finger in a particular area between and overlapping the optical detector and the screen of the portable electronic device. The portable electronic device may detect that the user's finger is present at the desired location (504) using one or more sensors. For example, in some implementations, a finger detection module may be included in the portable electronic device and may detect when the finger is present in the particular area between and overlapping the optical detector and the screen of the portable electronic device. The finger detection module may include a radar, a light sensor, or a touch sensor that may detect how far the finger is from the surface of the phone.

In response to detecting that the user's finger is present at the desired location (504), the portable electronic device may activate a display and optical detector of the portable electronic device and emit at least two light signals having different wavelengths (506). As explained above, emitters in the display configured to emit red, green, and blue wavelength signals may be used. For example, in some cases, red light LED emitters and green light LED emitters may emit red and green light, respectively. As shown in FIG. 3, light 340 from the display 315 (of the portable electronic device) may be emitted at a non-perpendicular angle with respect to the surface of the display 315 to improve the amount of the reflected signal detected by the camera 320.

In some implementations, the portable electronic device may also include other optical components to facilitate signal transmission and detection. For example, the portable electronic device may include one or more optical diffusers between the emitters and the user's skin. The optical diffusers may diffuse the emitted light signal so that a greater portion of the user's body part receives the emitted light. The portable electronic device may also include one or more of filters, lenses, and windows between the optical detector and the user's skin. The filters, lenses, and windows may facilitate collection of the reflected light signals by, e.g., guiding the reflected light to the optical detector or removing undesirable components of the signal.

As explained above, the emitted light may interact with the user's finger through a combination of reflection, scattering, and transmission. The optical detector in the portable electronic device may then detect light signals that have reflected back from the finger after interacting with the finger (508). The portable electronic device may continue to detect light signals for a determined period of time. When a camera is used as the optical detector, the camera may capture an image or a video over a particular time period to capture the reflected light signals. For instance, in some implementations when multiple emitters are used, an image or video may capture multiple reflected signals at the same time. In some implementations, when one emitter that sequentially outputs light at different wavelengths, multiple images or videos may sequentially capture the reflected signals.

In some implementations, signals detected by the optical detector may be conditioned for further processing. For example, the detected signals may be digitized, downconverted, downsampled, filtered, and converted to the frequency domain by performing a Fourier Transform prior to determining the physiological parameter values. In some cases, filters, such as adaptive filters used for color balancing, may be deactivated so as not to corrupt data that may be used to determine the physiological parameter values.

After detecting the reflected light signals and optionally performing signal conditioning, the portable electronic device may determine whether the SNR threshold has been satisfied (510). For example, the portable electronic device may determine whether the green and red reflected light signals each have a SNR ratio that satisfies, e.g., is greater than or equal to, the SNR threshold. The SNR threshold may be specified by the mobile application.

If the detected light signals do not have a SNR that satisfies the SNR threshold, the light emission levels of the emitters may be reconfigured (512) as described above with respect to the calibration procedure. For example, the red and green light emitters may be controlled to increase or decrease the emission levels of the emitted light to achieve improved and matching SNRs. After modifying the emitter configuration (512) as part of the calibration procedure, operations 506-510 may be repeated to obtain new reflected light signals.

Operations 506 to 512 may be repeated until reflection signals are received that satisfy the SNR threshold. In some implementations, if the SNR threshold is not satisfied after modifying the emitter configurations a determined number of times, the portable electronic device may output a message that measurements cannot be obtained at that time or that an error has occurred.

If the detected light signals have a SNR that satisfies the SNR threshold (510), the portable electronic device may then determine the physiological parameter values (514). For example, when determining the heart rate or respiration rate, the portable electronic device may perform a Fourier Transform on the reflection signal (if not already performed as part of the conditioning), and may determine the heart rate or respiration rate from the frequency content of the reflection signal.

When determining oxygen saturation levels, the portable electronic device may determine the AC and DC components of the red and green reflected light signals, as described above. After determining the physiological parameter values (514), the portable electronic device may output the values (516) using various suitable methods. For example, the portable electronic device may display one or more representations of the determined physiological parameter values on the display of the portable electronic device. The representations may include an alphanumeric value, a graphical representation, or even a color indication.

For example, if a user's heart rate or oxygen saturation levels are low such that the user's well-being is at risk, the display of the portable electronic device may emit a red color on a portion of the screen to indicate that the user's health is at risk. If the user's heart rate or oxygen saturation levels are within a normal range, as determined by doctors in the medical profession, the display of the portable electronic device may emit a green color on a portion of the screen to indicate that the user's health is good.

In some implementations, the portable electronic device may output an audio signal that indicates the determined physiological parameter values. In some implementations, the portable electronic device may transmit the determined physiological parameter values to a desired recipient such as, e.g., a second electronic device of the user, an electronic device of a family member or friend, or an electronic device associated with the user's doctor.

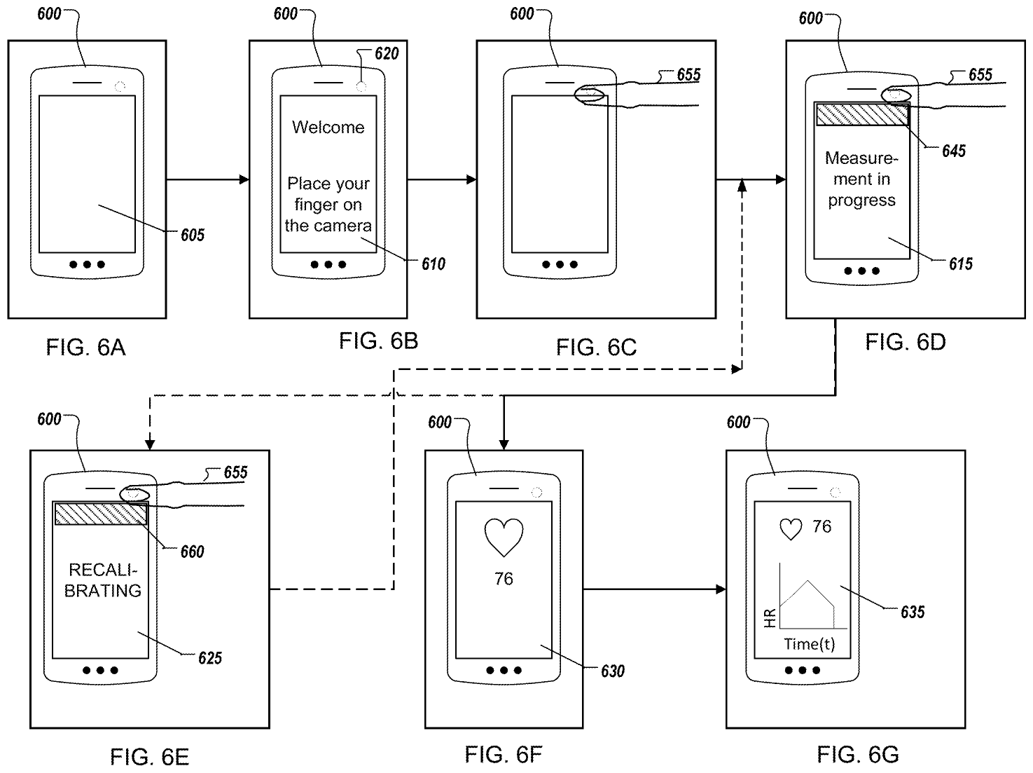

FIGS. 6A-6G depict screenshots corresponding to different phases of the process 500. In FIG. 6A, a mobile application for determining physiological parameter values has not been activated by the portable electronic device 600. The portable electronic device 600 may display a home screen or lock screen or content from other applications installed on the portable electronic device 600.

After receiving a signal to initiate the mobile application for determining physiological parameter values as described above in operation 502, the portable electronic device 600 may display a welcome screen 610. The welcome screen 610 may include one or more messages, e.g., “Welcome,” from the mobile application. The welcome screen 610 may also include instructions for the user to execute in order for the mobile application to perform the physiological measurements. For example, as shown in FIG. 6B, the message “Place your finger on the camera” is displayed by the portable electronic device 600.

The user may subsequently place a finger 655 on the camera 620, as shown in FIG.6C. The portable electronic device 600 may perform operations 506-510 described above with respect to FIG. 5. While performing these operations, the portable electronic device 600 may display a measurement in progress screen 615, as shown in FIG. 6D. The measurement in progress screen 615 may include a message such as “Measurement in progress” and may display a status bar 645 that indicates the progress made in performing the measurement operations.

If recalibration is needed, operation 512 may be performed and the portable electronic device 600 may display a recalibration screen 625 with a message indicating that a recalibration operation is being performed, as shown in FIG. 6E. The recalibration screen 625 may also include a recalibration status bar 660 that indicates the progress made in performing the recalibration operation.

If recalibration is not performed, the physiological parameter values are determined as described in operation 514. The determined values may then be displayed as part of operation 516. For example, as shown on screen 630 in FIG. 6F, a heart rate of the user may be displayed. In some cases, a history of the user's heart rate measurements may be displayed. For example, as shown in screen 635 in FIG. 6G, in addition to the heart rate “76” displayed in one portion of the screen 635, a graph may be displayed. The graph may depict a heart rate of the user over a period of time.

Although only a heart rate is shown in FIGS. 6F and 6G, multiple physiological parameter values may be displayed on a screen of the portable electronic device. For example, two or more of a heart rate, a respiration rate, an arterial oxygen saturation level, an iron level, a glucose level, a ketone level, a urea level, and a creatine kinase level may be displayed simultaneously by the portable electronic device. The display output may also be accompanied by an audio output, as described above with respect to operation 516.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, cellular networks, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can, in some cases, be excised from the combination, and the claimed combination may be directed to a sub-combination or variation of a sub-combination.

It should be understood that the phrase one or more of and the phrase at least one of include any combination of elements. For example, the phrase one or more of A and B includes A, B, or both A and B. Similarly, the phrase at least one of A and B includes A, B, or both A and B.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing can be advantageous.

What is claimed is:

1. A portable electronic device comprising:
a display comprising one or more emitters, the one or more emitters configured to transmit a first transmission signal at a first wavelength onto a body part of a user and a second transmission signal at a second wavelength onto the body part;
a camera configured to obtain one or more images comprising a first reflection signal in response to the first transmission signal being transmitted onto the body part and a second reflection signal in response to the second transmission signal being transmitted onto the body part; and
a processor configured to determine one or more physiological parameter values of the user using the one or more images obtained by the camera.

2. The portable electronic device of claim 1, wherein:
the first wavelength corresponds to a red light;
the second wavelength corresponds to a green light or a blue light; and
the body part of the user is a finger of the user.

3. The portable electronic device of claim 1, wherein:
the one or more emitters comprise:
a first emitter configured to emit the first transmission signal at the first wavelength, the first wavelength corresponding to a red light;
a second emitter configured to emit the second transmission signal at the second wavelength, the second wavelength corresponding to a green light; and
a third emitter configured to emit a third transmission signal at a third wavelength, the third wavelength corresponding to a blue light.

4. The portable electronic device of claim 1, wherein the one or more emitters comprise a first emitter configured to emit:
the first transmission signal corresponding to a red light at a first time;
the second transmission signal corresponding to a green light at a second time different from the first time; and
a third transmission signal corresponding to a blue light at a third time that is different from the first time or the second time.

5. The portable electronic device of claim 1, wherein:
the one or more emitters comprise one or more light emitting diodes;
the display is configured to display the one or more physiological parameter values; and
the one or more physiological parameter values comprise one or more values for a heart rate, a respiration rate, an arterial oxygen saturation level, an iron level, a glucose level, a ketone level, a urea level, and a creatine kinase level.

6. The portable electronic device of claim 1, comprising:
a detector configured to detect a presence of a body part of the user within a threshold distance of the display,
wherein the one or more emitters are configured to emit the first transmission signal and the second transmission signal in response to detecting the presence of the body part of the user within the threshold distance of the display.

7. The portable electronic device of claim 1, wherein:
the processor is configured to determine whether each of a signal to noise ratio of the first reflection signal and a signal to noise ratio of the second reflection signal satisfies a threshold; and
in response to determining that the signal to noise ratio of the first reflection signal does not satisfy the threshold, the one or more emitters are configured to retransmit the first transmission signal at a greater light emission level than an emission level of the prior transmission of the first transmission signal.

8. The portable electronic device of claim 1, wherein:
the processor is configured to determine whether each of a signal to noise ratio of the first reflection signal and a signal to noise ratio of the second reflection signal satisfy a threshold; and
in response to determining that the signal to noise ratio of the second reflection signal does not satisfy the threshold, the one or more emitters are configured to retransmit the second transmission signal at a greater light emission level than an emission level of the prior transmission of the second transmission signal.

9. A method of obtaining a physiological parameter value of a user using a portable electronic device, the method comprising:
receiving, by a processor in the portable electronic device, an initiation signal to initiate measurement of the physiological parameter value of the user;
in response to receiving the initiation signal to initiate the measurement of the physiological parameter value of the user, controlling one or more emitters in a display of the portable electronic device to transmit a first transmission signal at a first wavelength and a second transmission signal at a second wavelength onto a body part of the user;
obtaining one or more images comprising a first reflection signal in response to the first transmission signal being transmitted onto the body part and a second reflection signal in response to the second transmission signal being transmitted onto the body part;
determining, by the processor in the portable electronic device, the physiological parameter value using the one or more images; and
displaying, on a display of the portable electronic device, data indicative of the physiological parameter value.

10. The method of claim 9, comprising:
in response to receiving the initiation signal to initiate the measurement of the physiological parameter value of the user, determining whether the body part of the user is within a threshold distance of a camera of the portable electronic device; and
controlling the one or more emitters in the display of the portable electronic device to transmit the first transmission signal and the second transmission signal in response to determining that the body part of the user is within the threshold distance of the camera of the portable electronic device.

11. The method of claim 9, wherein:
the first wavelength corresponds to a red light;
the second wavelength corresponds to a green light or a blue light; and
the body part of the user is a finger of the user.

12. The method of claim 9, wherein, when multiple emitters are configured to emit light signals, obtaining the one or more images comprising the first reflection signal in response to the first transmission signal being transmitted onto the body part and the second reflection signal in response to the second transmission signal being transmitted onto the body part comprises obtaining a still image or a video that captures the first reflection signal and the second reflection signal.

13. The method of claim 9, wherein, when a single emitter is configured to emit light signals, obtaining the one or more images comprising the first reflection signal in response to the first transmission signal being transmitted onto the body part and the second reflection signal in response to the second transmission signal being transmitted onto the body part comprises obtaining multiple sequential images or a video that capture the first reflection signal and the second reflection signal.

14. The method of claim 9, wherein displaying the data indicative of the physiological parameter value comprises:
displaying data indicative of at least one of a heart rate, a respiration rate, an arterial oxygen saturation level, an iron level, a glucose level, a ketone level, a urea level, and a creatine kinase level.

15. The method of claim 9, comprising:
determining whether each of a signal to noise ratio of the first reflection signal and a signal to noise ratio of the second reflection signal satisfies a threshold;
in response to determining that the signal to noise ratio of the first reflection signal does not satisfy the threshold, retransmitting the first transmission signal at a greater light emission level than an emission level of the prior transmission of the first transmission signal; and
in response to determining that the signal to noise ratio of the second reflection signal does not satisfy the threshold, retransmitting the second transmission signal at a greater light emission level than an emission level of the prior transmission of the second transmission signal.

16. A non-transitory computer-readable storage medium comprising instructions, which when executed by one or more processors in a portable electronic device, cause the one or more processors to execute operations comprising:
receiving an initiation signal to initiate measurement of a physiological parameter value of a user;
in response to receiving the initiation signal to initiate the measurement of the physiological parameter value of the user, controlling one or more emitters in a display of the portable electronic device to transmit a first transmission signal at a first wavelength and a second transmission signal at a second wavelength onto a body part of the user;
obtaining one or more images comprising a first reflection signal in response to the first transmission signal being transmitted onto the body part and a second reflection signal in response to the second transmission signal being transmitted onto the body part;
determining the physiological parameter value using the one or more images; and
controlling a display of the portable electronic device to display data indicative of the physiological parameter value.

17. The non-transitory computer-readable storage medium of claim 16, wherein the operations comprise:
in response to receiving the initiation signal to initiate the measurement of the physiological parameter value of the user, determining whether the body part of the user is within a threshold distance of a camera of the portable electronic device; and
controlling the one or more emitters in the display of the portable electronic device to transmit the first transmission signal and the second transmission signal in response to determining that the body part of the user is within the threshold distance of the camera of the portable electronic device.

18. The non-transitory computer-readable storage medium of claim 16, wherein:
the first wavelength corresponds to a red light;
the second wavelength corresponds to a green light or a blue light; and
the body part of the user is a finger of the user.

19. The non-transitory computer-readable storage medium of claim 16, wherein displaying the data indicative of the physiological parameter value comprises:
displaying data indicative of at least one of a heart rate, a respiration rate, an arterial oxygen saturation level, an iron level, a glucose level, a ketone level, a urea level, and a creatine kinase level.

20. The non-transitory computer-readable storage medium of claim 16, wherein the operations comprise:
determining whether each of a signal to noise ratio of the first reflection signal and a signal to noise ratio of the second reflection signal satisfies a threshold;
in response to determining that the signal to noise ratio of the first reflection signal does not satisfy the threshold, retransmitting the first transmission signal at a greater light emission level than an emission level of the prior transmission of the first transmission signal; and
in response to determining that the signal to noise ratio of the second reflection signal does not satisfy the threshold, retransmitting the second transmission signal at a greater light emission level than an emission level of the prior transmission of the second transmission signal.
